(19) United States
(12) Patent Application Publication
(10) Pub. No.: US 2010/0145352 A1
(43) Pub. Date: Jun. 10, 2010

(54) HOOD METHOD AND DEVICE FOR ENDOSCOPIC SUBMUCOSAL DISSECTION

(76) Inventors: Kenneth Chang, Cerritos, CA (US); Richard W. Ducharme, Winston-Salem, NC (US)

Correspondence Address: BRINKS HOFER GILSON & LIONE/CHICAGO/COOK, PO BOX 10395, CHICAGO, IL, 60610, US

(21) Appl. No.: 12/631,200

(22) Filed: Dec. 4, 2009

Related U.S. Application Data
(60) Provisional application No. 61/120,378, filed on Dec. 5, 2008.
(60) Provisional application No. 61/120,220, filed on Dec. 5, 2008.

(51) Int. Cl. A61B 17/3205

(52) U.S. Cl. 606/110

(57) ABSTRACT

A medical device and a method of using such medical device for removing targeted tissue from a body lumen in a patient is presented. The method generally comprises placing the distal end of the device through a natural orifice in the patient to a location that is proximate to the targeted tissue; deploying a T-anchor fastened to a suture strand through the targeted tissue; deploying a loop anchor into the tissue of the body lumen spaced away from the targeted tissue, whereas the suture strand is slidably received by the loop anchor; applying tension to the suture strand; cutting the tissue at a predetermined depth around the periphery of the targeted tissue; and removing the targeted tissue along with the T-anchor from the body lumen. The tension applied to the targeted tissue maintains the targeted tissue in a raised position and/or allows the physician to manipulate the targeted tissue relative to the tissue that is proximate to it.

This application claims the benefit of U.S. Provisional Application Ser. No. 61/120,378 filed on Dec. 5, 2008, entitled “HOOD METHOD FOR ENDOSCOPIC SUBMUCOSAL DISSECTION,” and U.S. Provisional Application Ser. No. 61/120,220 filed on Dec. 5, 2008, entitled “TISSUE ANCHORS FOR PURSE-STRING CLOSURE OF PERFORATIONS,” the entire contents of which are incorporated herein by reference.

### FIELD

This disclosure relates generally to a method for lifting and manipulating tissue endoscopically, such as for performing an endoscopic submucosal dissection (ESD) or endoscopic mucosal resection (EMR) of tissue.

### BACKGROUND

Diagnostic and therapeutic gastrointestinal endoscopy is a common technique used to gain access to the digestive tract for the purpose of removing tissue. Endoscopic mucosal resection (EMR) is one method of performing the biopsy necessary to obtain tissue for pathology examination. An EMR procedure also may be used for curative purposes, such as to remove sessile benign tumors and intramucosal cancers. In fact, EMR is a well-accepted treatment for early gastric cancer.

It has been discovered that during curative removal of a mucosal lesion, it is desirable to remove the lesion as one piece. If the lesion is removed as multiple fragments, it is believed that rates of local tumor recurrence may be increased. In addition, medical examination and assessment using fragmented tissue samples can be more difficult to perform than a similar assessment done with a single piece of tissue.

During an EMR procedure, it may be desirable to mark and subsequently resect a portion of tissue surrounding a lesion to help ensure that the lesion can be completely removed as a single piece. In addition to removing the mucosal tissue, a portion of the submucosa also may be removed. It is often difficult to tell if the entire lesion has been removed because of unclear delineation between the lesion and the surrounding tissue. Often a larger section of tissue that includes the lesion and a portion of the surrounding tissue is removed. One drawback to EMR is that this procedure is generally not recommended for large lesions, e.g., lesions that are greater than about two centimeters in diameter.

More recently, a technique called endoscopic submucosal dissection (ESD) has been described in which mucosal lesions are removed by the dissection of the submucosal tissue under the lesion using an incision device, such as an endoscopic knife. When compared to EMR, this ESD procedure facilitates the resection of larger lesions and yields improved removal of the lesion as one piece. However, one drawback to this technique is that the peripheral boundary of the lesion becomes difficult to ascertain as the tissue is surgically cut.

In view of the drawbacks of current technology, it is desirable to develop apparatus and methods for ESD and EMR procedures that may efficiently remove a mucosal or submucosal lesion as a single piece.

### SUMMARY

The present disclosure provides an apparatus or device, as well as a method of using such device for removing targeted tissue from a body lumen in a patient.

One embodiment of the device, constructed in accordance with the teachings of the present disclosure, the device is generally characterized by a suture strand, a T-anchor, a loop anchor, a flexible delivery needle, and a stylet. The stylet is in communication with the T-anchor and loop anchor such that it can cause the T-anchor to exit the needle in order for the physician to couple the T-anchor to the targeted tissue. The stylet can also cause the loop anchor to exit the needle in order for the physician to couple the loop anchor to the surrounding tissue in the body lumen.

One embodiment of the method, constructed in accordance with the teachings of the present disclosure, generally comprises placing the distal end of a endoscope through a natural orifice in a patient to a location that is proximate to the targeted tissue; deploying a T-anchor fastened to a suture strand through the targeted tissue; deploying a loop anchor into the tissue of the body lumen spaced away from the targeted tissue whereas the suture strand is slidably received by the loop anchor; applying tension to the suture strand to lift the targeted tissue; cutting the tissue at a predetermined depth around the periphery of the targeted tissue; and removing the targeted tissue along with the T-anchor from the body lumen. Preferably, the loop anchor is deployed into the tissue of the body lumen generally opposite the targeted tissue.

The tension applied to the targeted tissue maintains the targeted tissue in a raised position relative to the tissue proximate to the targeted tissue and/or allows the physician to manipulate the targeted tissue, thereby, rendering the targeted tissue more visible during the surgical procedure. The amount of tension applied to the suture strand may be adjusted as the targeted tissue is being cut.

In another embodiment of the present disclosure, an injection needle may be optionally inserted into a section of tissue proximate to the targeted tissue. A fluid can then be injected beneath the section of tissue proximate to the targeted tissue in order to raise the targeted tissue.

According to another aspect of the present disclosure, the targeted tissue to be removed is a lesion that is confined within the mucosa tissue, submucosa tissue, or a combination thereof. Incision markings may be placed in the mucosa tissue around the periphery of the targeted tissue if desired. An injection needle is inserted into either the mucosa tissue or submucosa tissue and a fluid is injected. This fluid preferably comprises a saline solution or sodium hyaluronate. The injected fluid forms a fluid pocket between the muscularis propria tissue and the submucosa tissue, between the submucosa tissue and the mucosa tissue, or entirely within the submucosa tissue.

According to yet another aspect of the present disclosure, tension is applied to the suture strand either by hand or using a device, instrument, or a robot. Similarly, the adjustment of the tension may be accomplished in the same or similar manner.

The cutting of the tissue around the periphery of the targeted tissue is done using an electrified or mechanical endoscopic cutting instrument. The cutting instrument may have a hollow end or ceramic tip in order to assist in cutting only to a predetermined depth. After the periphery of the target tissue has been completely incised, the targeted tissue may be removed using a retrieval device, such as a snare or forceps. Optionally, the loop anchor in the body lumen may also be removed.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a schematic representation of a method or method according to one embodiment of the present disclosure used to remove targeted tissue from a bodily lumen in a patient;

FIG. 2A is a side view of a method step that may be used according to the teachings of the present disclosure;

FIG. 2B is a side view of an optional method step that may be used according to the teachings of the present disclosure;

FIG. 2C is a side view of another optional method step that may be used according to the teachings of the present disclosure;

FIG. 2D is a side view of another method step that may be used according to the teachings of the present disclosure;

FIG. 2E is a side view of another method step that may be used according to the teachings of the present disclosure;

FIG. 2F is a side view of another method step that may be used according to the teachings of the present disclosure;

FIG. 2G is a side view of another method step that may be used according to the teachings of the present disclosure;

FIG. 2H is a side view of another optional method step that may be used according to the teachings of the present disclosure;

FIG. 2I is a side view of another method step that may be used according to the teachings of the present disclosure;

FIG. 3 is a front view of one embodiment of a tissue anchor constructed in accordance with the teachings of the present disclosure;

FIG. 4 is a cross-sectional view taken about the line 4-4 in FIG. 3;

FIG. 5A is a cross-sectional view of a medical device constructed according to the teachings of the present disclosure;

FIG. 5B is another cross-sectional view of the medical device of FIG. 5A engaged in the deployment of the T-anchor; and

FIG. 5C is yet another cross-sectional view of the medical device of FIG. 5A engaged in the deployment of the loop anchor.

### DETAILED DESCRIPTION

The following description is merely exemplary in nature and is in no way intended to limit the present disclosure or its application or uses. For example, while endoscopy and the use of an endoscope are described to illustrate the present disclosure, other types of procedures and their associated devices, such as laparoscopy are contemplated to be within the scope of the disclosure. Likewise, procedures other than ESD and EMR that require tissue to be raised or lifted may also employ the teachings of the present disclosure. It should be understood that throughout the description and drawings, corresponding reference numerals indicate like or corresponding parts and features.

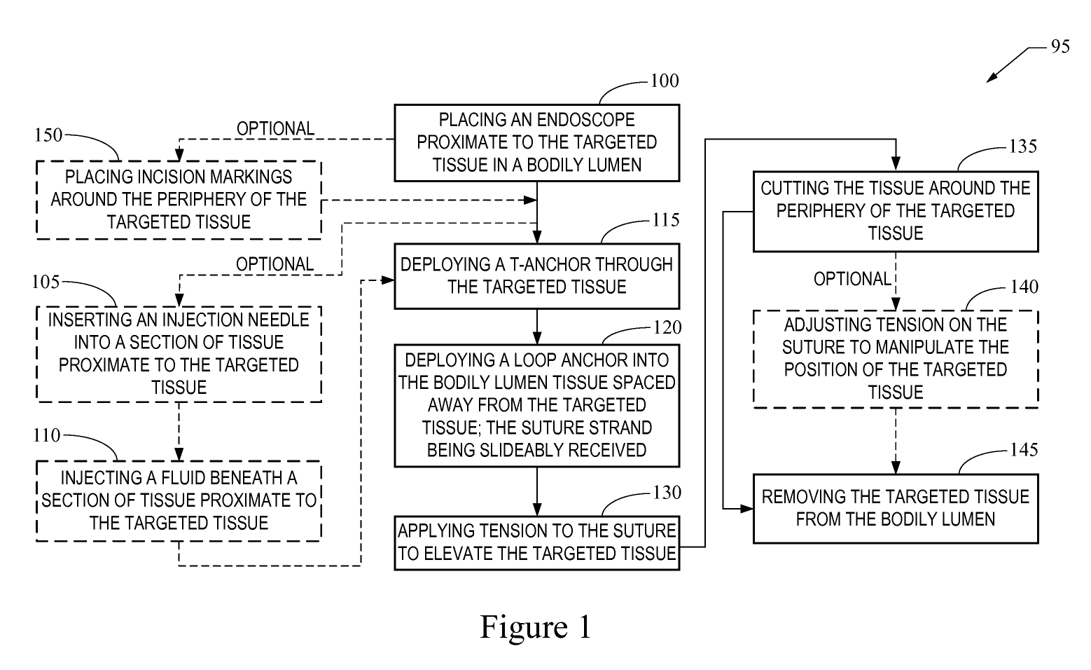

The present disclosure generally provides a medical device and a method of using the medical device for removing targeted tissue from a body lumen in a patient. Referring to FIG. 1, the method comprises placing 100 the distal end of a endoscope through a natural orifice in the patient to a location that is proximate to the targeted tissue; deploying 115 a T-anchor fastened to a suture strand through the targeted tissue; deploying 120 a loop anchor into the tissue of the body lumen spaced away from the targeted tissue, whereas the suture strand is slidably received by the loop anchor; applying 130 tension to the suture strand; cutting 135 the tissue at a predetermined depth around the periphery of the targeted tissue; and removing 145 the targeted tissue from the body lumen. Preferably, the loop anchor is deployed into the tissue of the body lumen generally opposite the targeted tissue, but the loop anchor may be circumferentially spaced from the target any degree. The endoscope may be a single channel or a multi-channel endoscope.

The tension applied to the targeted tissue maintains the targeted tissue in a raised position relative to the tissue proximate to the targeted tissue and/or allows the physician to manipulate the targeted tissue, thereby, rendering the targeted tissue more visible during the surgical procedure. The amount of tension applied to the suture strand may be adjusted as the targeted tissue is being cut.

The method may optionally include the steps of inserting 105 an injection needle into a section of tissue proximate to the targeted tissue; and injecting 110 a fluid beneath a section of tissue proximate to the targeted tissue in order to raise the targeted tissue. Alternatively, the method may optionally include the steps of disposing 155 at least a portion of a balloon beneath the targeted tissue; and inflating 160 the balloon in order to raise the targeted tissue. These optional steps may assist the physician in be able to ascertain the periphery of the targeted tissue.

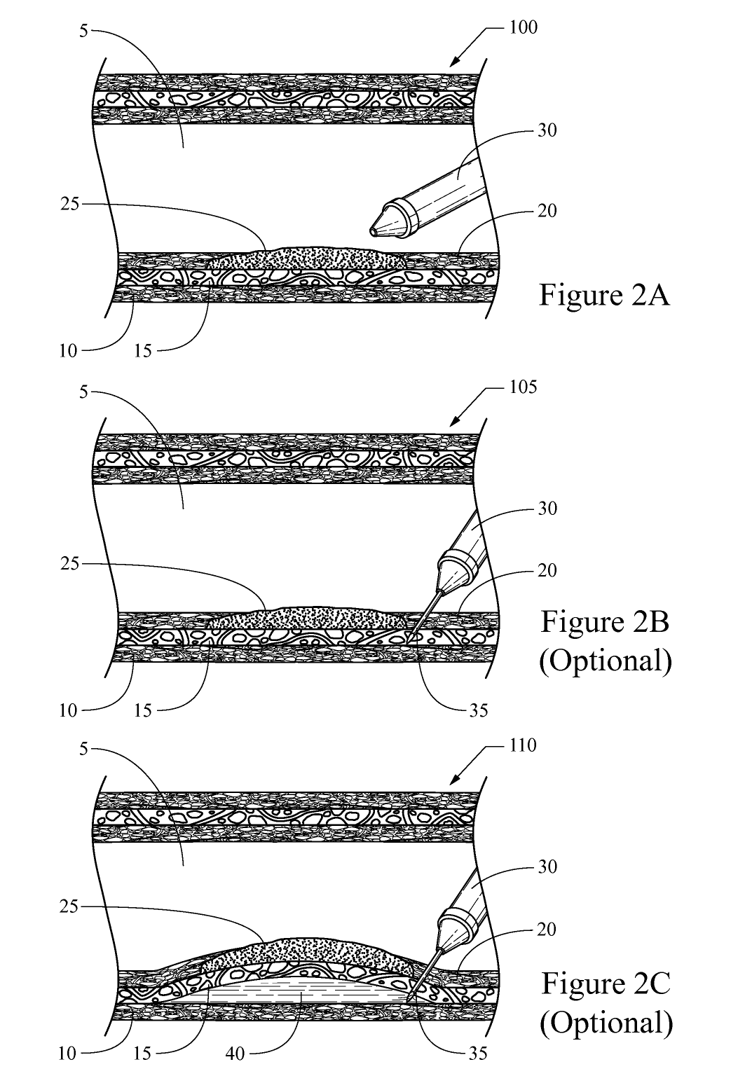

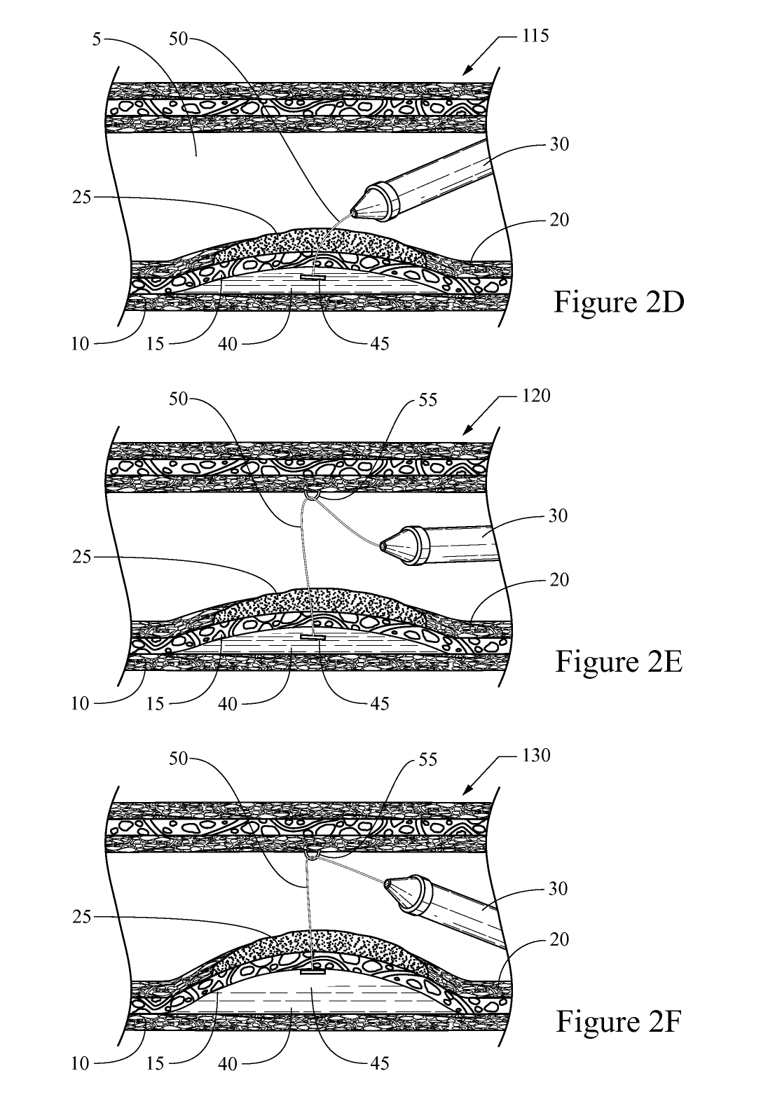

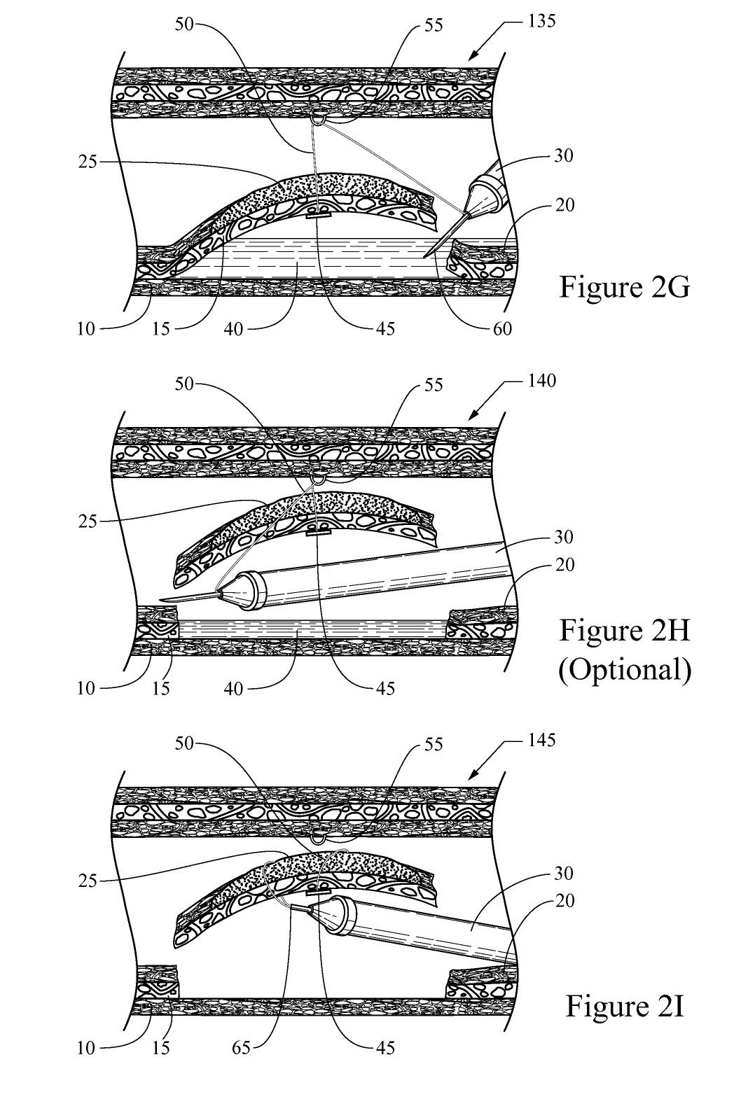

Referring now to FIG. 2A, an endoscope 30 is maneuvered towards the targeted tissue 25 using general endoscopy techniques that are well known to those skilled in the art. For example, the endoscope 30 may be maneuvered into a patient's body lumen 5, such as but not limited to the mouth, down through the esophagus, stomach, and duodenum, to a position that is proximate to the targeted tissue 25. The targeted tissue 25 may comprise a lesion, for example, a growth that is indicative of gastric cancer. This lesion or growth may be fully or partially confined within the mucosal tissue 20 and/or the submucosa tissue 15 with the muscularis propria tissue 10 residing beneath the submucosa tissue 15.

The distal region of an endoscope 30 preferably comprises a working channel and auxiliary lumen with the working channel sized to accommodate various components used to perform an endoscopic submucosa dissection (ESD) or endoscopic mucosal resection (EMR) procedure, such as a needle, needle knife, anchors, suture, and the like. One skilled in the art will realize that the endoscope 30 may comprise any number of lumens or channels to achieve the objects of the present disclosure. The endoscope 30 may also comprise optical elements, which employ fiber optic components for illuminating and capturing an image distal to the endoscope 30 for viewing by the physician performing the procedure. It will also be recognized that other visualization techniques may be employed including catheter-based fiber optic systems, fluoroscopy, ultrasound or the like. For example, a needle or other endoscopic tool can have markings designed for viewing under fluoroscopy, and the distal end of the needle can have a surface of enhanced ultrasonic reflectivity, such by being roughened, having dimples or other incongruities, or having embedded particles. In the latter case, the endoscope 30 may comprise an ultrasound-enabled endoscope system (EUS) permitting placement of the tool under EUS guidance, thereby facilitating placement within or between layers of tissue (e.g. within the submucosa layer) or preventing puncture of other organs or tissue behind the selected body wall.

Once the endoscope 30 is positioned proximate to the targeted tissue 25, the tending physician may examine the lesion and determine whether or not incision markings are necessary to help define the boundary of the targeted tissue 25. Placing incision markings 150 around the periphery of the targeted tissue 25 is an optional step in the method, which can be omitted when the targeted tissue 25 can readily be distinguished from the surrounding mucosa tissue 20. If the peripheral boundary of the targeted tissue 25 is not readily discernible, then a needle knife may then be advanced from the endoscope 30 and used to engage the mucosa tissue 20 to create markings around the periphery of the targeted tissue 25. High frequency current may be applied to the needle knife tip to create these markings. Such methods for creating markings are well known to those skilled in the art of endoscopic surgery.

Referring now to FIGS. 2B and 2C the targeted tissue 25 may be lifted or raised with respect to the muscularis propria tissue 10 to facilitate the removal of the targeted tissue 25. The targeted tissue 25 may optionally be raised by injecting a fluid 40 (FIG. 2C), such as physiological saline solution or sodium hyaluronate, through a needle 35 that is inserted into the mucosa tissue 20 and/or submucosa tissue 15 proximate to the targeted tissue 25 (FIG. 2B). The targeted tissue 25 may also be optionally raised by disposing at least a portion of a balloon beneath the targeted tissue, followed by inflating the balloon (not shown), as described in U.S. patent application Ser. No. 11/786,274 filed Apr. 11, 2007, the entire contents of which are hereby incorporated by reference. One skilled in the art will recognize that the needle 35 and a needle knife or any other surgical tool that will be later used to cut the tissue may be disposed and advanced through the same or different lumens of the endoscope 30. For example, the needle 35 may be disposed within an auxiliary lumen, while a needle knife is advanced through the working channel of the endoscope 30. Alternatively, the needle knife may be disposed within a hollow interior region of the needle 35, and the fluid 40 may be injected through the needle 35 so that it flows around the needle knife.

As shown in FIG. 2C according to one embodiment of the present disclosure, the fluid 40 injected into the submucosa tissue 15 lifts or raises the targeted tissue 25 above the underlying muscularis propria tissue 10, by forming a fluid 40 filled pocket between the muscularis propria tissue 10 and the submucosa tissue 15. The fluid 40 filled pocket according to another aspect of the present disclosure may also be formed entirely within the submucosa tissue 15 or between the mucosa tissue 20 and submucosa tissue 15. Optionally, a balloon may be at least partially inserted beneath the targeted tissue 25 and inflated in order to raise the targeted tissue 25. The raising of the targeted tissue 25 helps to facilitate its removal during an EMR or ESD procedure. This step is optional and may be used in conjunction with the following steps or eliminated as the following steps can also be used to raise the targeted tissue 25 above the surrounding mucosa tissue 20. One skilled in the art will understand that that other means of raising the targeted tissue, such as the use of a lever portion on the tip of the endoscope configured to lift the targeted tissue as described in co-pending U.S. patent application Ser. No. 11/849,736, filed on Sep. 4, 2007, which is hereby incorporated in its entirety by reference, may additionally be employed without exceeding the scope of the present disclosure.

Referring now to FIG. 2D, a T-anchor 45 is deployed through the targeted tissue 25 using any technique known to one skilled in the art of surgery. A T-anchor 45 typically comprises an anchor bar having a longitudinal configuration and a suture strand 50 attached to the bar and extending laterally from the bar. An introducer needle can be used to pass the anchor bar through the targeted tissue 25 and to deploy the anchor bar on the far side or underneath the targeted tissue 25. Guidance under EUS, fluoroscopy, visual markings, or using other depth indicating means may be employed, especially when a fluid filled pocket is not formed. The suture strand 50 extends through the targeted tissue 25, and has a bolster on the near side of the targeted tissue 25. The bolster is slidable along with the suture strand 50. As the suture strand 50 is pulled and the bolster is pushed, the anchor bar seats against the far-side of the targeted tissue 25. A knot tied in the suture on the proximal side of the bolster maintains the T-anchor 45 in an operative position in the targeted tissue 25.

The anchor bar of the T-anchor 45 can be made out of any material that is compatible with the human body, including but not limited to plastics, such as polyethylene, and metals, such as stainless steel. The suture strand 50 may be comprised of any material known to one skilled in the art. Materials, such as 2-0 silk, 2-0 Ti-Cron, 4-0 polypropylene, 5-0 polypropylene, 6-0 polypropylene, and 7-0 polypropylene are preferred. Further details regarding T-anchors are disclosed in U.S. Pat. No. 5,123,914 issued Jun. 23, 1992, the entire contents of which are herein incorporated by reference. One skilled in the art will recognize that any other type of tissue anchoring members, such as an anchor, staple, clip, hook, or tack that can be secured or fastened to the targeted tissue may be used (see, for example, the tacks disclosed in U.S. patent application Ser. No. 12/428,226 filed on Apr. 22, 2009). Preferably the suture strand 50 is selected with the appropriate level of abrasion resistance to withstand the movement of the suture strand 50 through the loop anchor 55.

Referring now to FIG. 2E, a loop anchor 55 is deployed into the tissue of the bodily lumen 5 opposite the targeted tissue 25 using any technique known to one skilled in the art of surgery. In general, a loop anchor 55 has a crossbar that is inserted into the tissue, and defines a loop or at least one cavity capable of slidably receiving a suture strand 50. Further details of this type of anchor and other types of anchors may be found in U.S. patent application Ser. No. 11/946,565 filed Nov. 25, 2007, the entire contents of which are herein incorporated by reference. One skilled in the art will recognize that any other type of anchor, staple, clip or tack, in which the suture strand 50 can be slidably received may be used. For example, a tack such as those disclosed in U.S. Patent Application No. 61/047,293 filed on Apr. 23, 2008, the entire contents of which are hereby incorporated by reference, might be employed.

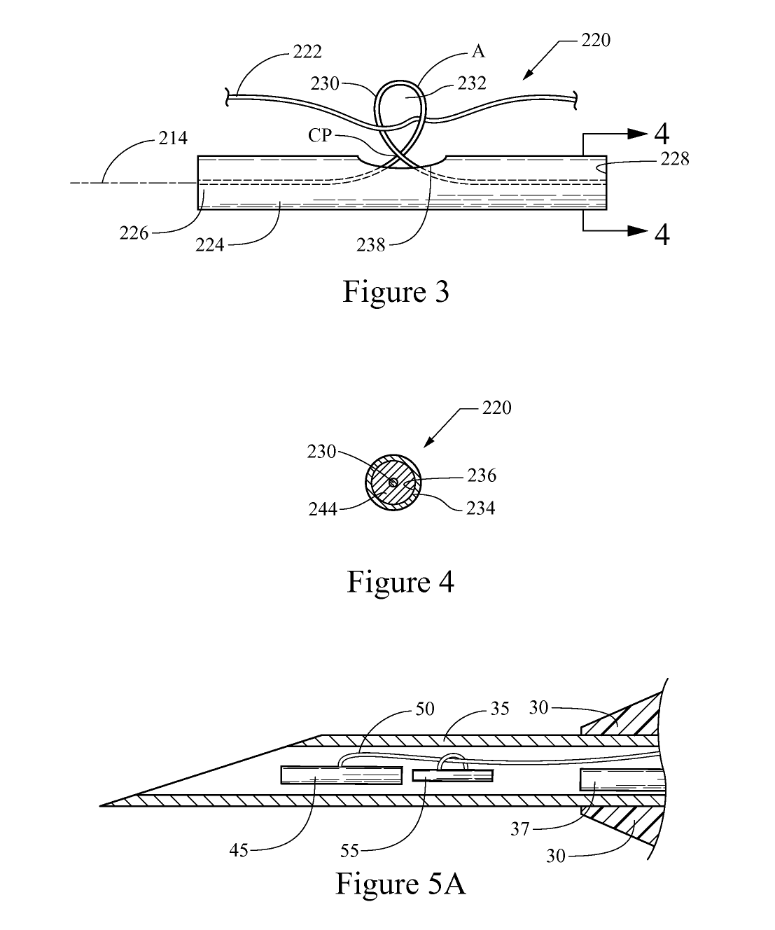

The currently preferred loop anchor 220 is shown in FIGS. 3-4. The anchor 220 generally includes a crossbar 224 having opposing ends 226 and 228 and defining a longitudinal axis 214. The crossbar 224 is preferably elongated, but may take any form suitable for connecting the suture 222 to the bodily wall 212. A strand 230 is connected to the crossbar 224 and is configured to form a loop 232. As best seen in FIG. 4, the crossbar 224 is constructed of a cannula having a tubular wall 234 defining a lumen 236. An elongated aperture 238 is formed in the tubular wall 234, and the strand 230 passes through the aperture 238. The ends of strand 230 are secured within the lumen 236 of the cannula by welds 244. It will be recognized by those skilled in the art that the strand 230 may be secured to the crossbar 224 using any now known or hereinafter developed attachment means, including mechanical fasteners, adhesives or various welding or soldering techniques.

The strand 230 is preferably formed from a metal wire, including single filament and multi-filament wires, and wound and braided wires, although the strand 230 can have other constructions such as suture material, plastic strings, rope and the like. As best seen in FIG. 3, the strand 230 is structured to include a revolution thereby defining a loop 232 through which the suture 50 passes. The loop 232 is positioned longitudinally in-line with the elongated aperture 238 so that it projects through the aperture 238 and away from the longitudinal axis 214. Accordingly, it will be seen that the strand 230 and its loop 232 are flexible and may adjust its shape and orientation based on how the suture 50 is being tensioned. The size of the elongated aperture 238 and the flexibility of the strand 230 allow the loop 232 to travel longitudinally along the length of the strand 230. The loop 232 defines an apex A which is preferably located about 0.35 mm or greater away from the crossbar 224. The loop 232 also defines a cross-point CP where the ends of the strand 230 cross each other. The cross-point CP is preferably positioned radially outside the outer surface of the crossbar 224 including radially outside the side walls of the aperture 238, but also preferably as close to the crossbar 224 as possible. The aperture 238 preferably extends a longitudinal distance in a range of about 0.4 mm to about 3.0 mm, while the crossbar 224 typically has a length in the range of about 3.0 mm to about 10.0 mm. The strand preferably has a diameter less than about 50% of a diameter of the crossbar 224, and most preferably less than about 35%. The strand 230 preferably has a diameter in the range of about 0.20 mm to about 0.35 mm, and most preferably about 0.0254 mm. The crossbar 224 preferably has a diameter in the range of about 0.5 mm to about 1.0 mm, and most preferably about 0.8 mm. The strand 230 may be coated with a low-friction material such as known plastic or hydrophilic coatings.

This construction of the tissue anchor 224 and its loop 232 allows the suture 50 to be tensioned and slid through the loop 232 relative to the crossbar 224 while preventing the suture 50 from engaging the crossbar 224 or the edges defined by the elongated aperture 238. That is, no matter which direction the ends of the suture 50 are pulled or slid relative to the crossbar 24, the wire 230 and its loop 232 will serve as a barrier between the suture 50 and the canula 224 to prevent any undesired abrasion therebetween. Generally, the strand 230 has a length and the location of the apex A of the loop 232 are such that the loop 232 is sized to project through the bodily wall 212 when embedded therein, allowing reliable tensioning of the suture 50 and preventing abrasion of the tissue. One skilled in the art will understand that other loop anchor designs may be utilized without exceeding the scope of the present disclosure.

Referring now to FIG. 2F, a predetermined amount of tension is applied to the suture strand 50 by the physician or operator. The application of tension to the suture strand 50 assists in raising the targeted tissue 25 above the surrounding mucosa tissue 20, thereby, making the periphery of the targeted tissue 25 easier to visualize. This tension may be applied and manipulated by hand, using a device or instrument, or by a robot. The tension can be adjusted and maintained using a suture tensioning device, such as the one disclosed in co-pending U.S. patent application Ser. No. 12/630,373 filed on Dec. 3, 2009, the entire contents of which are hereby incorporated by reference.

Optionally, after the T-anchor 45 and loop anchor 55 have been positioned, the endoscope 30 may be removed and reintroduced to a position proximate to the suture strand 50, e.g. along side the suture strand 50. This optional step allows the physician additional freedom or latitude in approaching the targeted tissue 25 in order to make an incision; as well as insuring that the endoscope 30 does not affect the applied tension and makes the accessory channel of the endoscope 30 more easily accessible.

Referring now to FIG. 2G, after the targeted tissue 25 has been sufficiently raised and appropriate tension has been applied to the suture strand 50 connected to the T-anchor 45, the creation of a mucosal or submucosal incision begins. A cutting instrument (e.g., needle knife) 60 or another electrified or mechanical endoscopic cutting instrument, such as a scalpel or the like, may be advanced distally through a channel or lumen in the endoscope 30. The incision may be made circumferentially around the targeted tissue 25 using the needle knife 60, as depicted by FIGS. 2G-2I. An electrosurgical generator (not shown) may be coupled to the needle knife 60 to provide the electrical energy necessary to incise the cut tissue. The incision is preferably performed at a predetermined distance into or through the submucosa 15 tissue and at a predetermined angle with respect to muscularis propria tissue 10

As the tissue is cut, the operator may optionally, as shown in FIG. 2H, adjust the tension applied to the suture strand 50 in order to maintain the targeted tissue 25 in a raised position or preferably to manipulate (e.g., raise or lower) the targeted tissue 25 relative to the surrounding mucosa tissue 20. Manipulating the targeted tissue 25 via the use of tension applied to the suture strand 50 is advantageous because it allows the physician to more easily determine the periphery of the targeted tissue 25 through which the incision is being made. This step makes cutting the tissue safer and faster because the physician more readily can see where he wants to make the incision. The ability to see the periphery of the targeted tissue 25 may reduce the time necessary for the medical procedure from more than 4 hours to about less than 1 hour. If the tension applied to the suture strand 50 does not allow the physician to manipulate the targeted tissue 25, it is possible that any fluid 40 released from the fluid 40 filled pocket (if present) upon the start of the incision may obscure the periphery of the targeted tissue 25, thereby, complicating the removal of the targeted tissue 25.

The needle knife 60 may be fabricated using any electrically conductive material known to one skilled in the art, including but not limited to stainless steel. Alternatively, the needle knife may be fabricated from a shape memory alloy such as nitinol, as described in co-pending U.S. patent application Ser. No. 11/729,402 filed Mar. 28, 2007, the entire contents of which are hereby incorporated by reference. Optionally, the needle knife 60 may comprise a non-conductive portion at its tip, such as a hollow or ceramic region, that will help prevent the needle knife from cutting too far into the tissue. The use of other such safety mechanisms will be apparent to one skilled in the art.

Referring now to FIG. 2I, once the incised targeted tissue 25 is substantially separated from the surrounding mucosa tissue 20 and submucosa tissue 15, the needle knife 60 may be withdrawn and the tension applied to the suture strand 50 released. A retrieval device 65, such as a snare or forceps, then may be advanced through the auxiliary or working lumen of the endoscope 30 to subsequently remove the incised targeted tissue 25 along with its imbedded T-anchor 45 in substantially one piece. The removal of the targeted tissue 25 in substantially one piece assists in reducing the likelihood of the lesion or growth locally reoccurring in the patient. The endoscope 30 then may be removed from the patient to complete the procedure. Optionally, the loop anchor 55 may be left imbedded in the body lumen 5 or be removed by any technique known to one skilled in the art.

If desired, flushing fluid may be provided near the targeted tissue 25 at any time during the EMR or ESD procedure. For example, the flushing fluid may be delivered through the auxiliary lumen or working channel of the endoscope 30, and may be delivered around needle 35 and/or needle knife 60, as described in co-pending U.S. patent application Ser. No. 11/747,570 filed May 11, 2007, which is hereby incorporated by reference in its entirety.

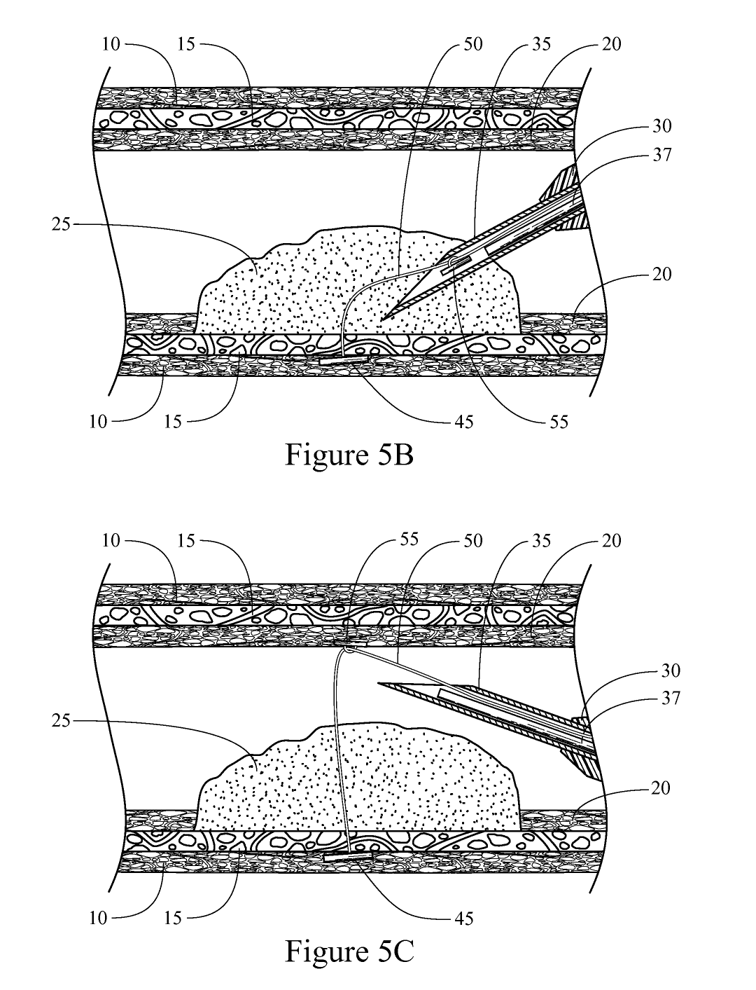

A preferred device or apparatus 1 used to assist and operator in distinguishing and/or lifting the targeted tissue 25 from surrounding tissue 20 in a body lumen that is adjacent thereto when the physician performs the method described above for removing the targeted tissue 25 from other tissue 10, 15, 20 present in a body lumen is shown in FIG. 5A. This device or apparatus 1 is characterized by a suture strand 50, a first anchor member (e.g., T-anchor) 45, a second anchor member (e.g., loop anchor) 55, a flexible delivery needle 35, and a stylet 37.

The suture 50 is defined by having a distal and proximal end. The first anchor member 45 has a bar connected thereto and is fixed to the distal end of the suture strand 50. The second anchor member 55 defines a loop through which the suture strand 50 passes in order to position the second anchor member 55 proximate to the first anchor member 45. The second anchor member 55 has a bar and a flexible loop that projects away from the bar. The first anchor member 45 is positioned distal to the second anchor member 55 within the needle 35 with the suture strand passing through the loop of the second anchor member within the needle.

The flexible delivery needle 35 defines at least one passageway that is sized to slidably receive the suture strand 50, the first anchor member 45, and the second anchor member 55. The distal end of the needle 35 is shaped to allow the needle 35 to pierce the targeted tissue 25. Finally, the stylet 37 is sized to be slidably received by the delivery needle 35.

Referring now to FIGS. 5A-5C, the stylet 37 is also in communication with the second anchor member 55 such that the stylet 37 can cause the first anchor member 45 to exit the needle 35 in order for the physician to couple the first anchor member 45 to the targeted tissue 25 (FIG. 5B). The stylet can also cause the second anchor member 55 to exit the needle 35 (FIG. 5C). When tension is applied to the suture strand 50, the suture strand 50 slides through the loop of the second anchor member 55 to move the first anchor member 45, thereby assisting the physician to lift the targeted tissue 25 and/or distinguish between the targeted tissue 25 and other tissue 20.

The medical device 1 further may further comprise a housing 30 that can be manipulated by the physician. The housing 30 has at least one passageway sized to slidably receive the delivery needle 35. An example of a housing 30 is an endoscope. In addition, the device 1 may also includes a retrieval device 65 sized to be slidably received by a passageway of the housing 30. The retrieval device 65 is capable of engaging the targeted tissue 25 to further separate the targeted tissue 25 from other tissue 20.

The medical device 1 may also include a cutting instrument 60 sized to be slidably received in one passageway of either the needle 35 or the housing 30. The cutting instrument 60 is capable of being manipulated by the physician to separate the targeted tissue 25 from other tissue 20. The cutting instrument 60 may have a hollow end or ceramic tip in order to assist in cutting only to a predetermined depth.

The foregoing description of various embodiments of the disclosure has been presented for purposes of illustration and description. It will be appreciated that the apparatus and methods described herein above may be used to treat various types of lesions, e.g., large superficial tumors and intraepithelial neoplasms, in virtually any body cavity, such as the stomach, esophagus and colon. It is not intended to be exhaustive or to limit the disclosure to the precise embodiments disclosed. Numerous modifications and variations are possible in light of the above teachings. The embodiments discussed were chosen and described to provide the best illustration of the principles of the disclosure and its practical application to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

It will be recognized by those skilled in the art that, while the methods described in this disclosure generally include placing the tissue devices in tissue through an internal bodily lumen, it will be recognized that these systems, devices and methods may be used on any layer of material (e.g. fabrics, cloth, polymers, elastomers, plastics and rubber) that may or may not be associated with a human or animal body and a bodily lumen. For example, the systems, devices and methods can find use in laboratory and industrial settings for placing devices through one or more layers of material that may or may not find application to the human or animal body, and likewise closing holes or perforations in layers of material that are not bodily tissue.

What is claimed is:

1. A method for removing targeted tissue from a body lumen in a patient, the method comprising the steps of:
placing the distal end of an endoscope through a natural orifice in the patient to a location that is proximate to the targeted tissue;
deploying a T-anchor fastened to a suture strand through the targeted tissue;
deploying a loop anchor into the tissue of the body lumen spaced away from the targeted tissue, whereas the suture strand is slidably received by the loop anchor;
applying tension to the suture strand;
cutting the tissue at a predetermined depth around the periphery of the targeted tissue; and
removing the targeted tissue along with the T-anchor from the body lumen;
wherein the tension applied to the targeted tissue maintains the targeted tissue in a raised position relative to the tissue proximate to the targeted tissue.

2. The method of claim 1, further comprising the steps of either
inserting an injection needle into a section of tissue proximate to the targeted tissue; and
injecting a fluid beneath the section of tissue proximate to the targeted tissue in order to raise the targeted tissue; or
disposing at least a portion of a balloon beneath the targeted tissue; and
inflating the balloon in order to raise the targeted tissue.

3. The method of claim 1, wherein during the step of deploying the loop anchor, the loop anchor is deployed into the tissue of the body lumen generally opposite the targeted tissue.

4. The method of claim 1, wherein during the step of deploying the loop anchor, the loop anchor comprises a crossbar and a strand, the crossbar having first and second opposing ends and defining a longitudinal axis, the crossbar being defined by a tubular wall having an aperture between the first and second ends, the strand having first and second opposing ends connected to the first and second opposing ends of the crossbar, respectively, the strand making a revolution to define a loop, the strand and its loop projecting through the aperture and away from the longitudinal axis, the loop sized to slidably receive the suture therein and protect the suture from abrasion against the crossbar.

5. The method of claim 1, further comprising the step of adjusting the tension applied to the suture strand as the tissue is cut.

6. The method of claim 1, wherein the targeted tissue to be removed is a lesion confined within one group selected from mucosa tissue, submucosa tissue, and a combination thereof.

7. The method of claim 2, wherein the step of inserting an injection needle is done by inserting the needle into one selected from the group of mucosa tissue or submucosa tissue; and
wherein the step of injecting fluid forms a fluid filled pocket located within one selected from the group of between the muscularis propria tissue and the submucosa tissue; between the submucosa tissue and the mucosa tissue; and entirely within the submucosa tissue.

8. The method of claim 1, further comprising the step of placing incision markings in the mucosa tissue around the periphery of the targeted tissue.

9. The method of claim 1, wherein the steps of applying the tension on the suture strand may be accomplished using one selected from the group of by hand, a device, an instrument, and a robot.

10. The method of claim 5, wherein the step of adjusting the tension on the suture strand may be accomplished using one selected from the group of by hand, a device, an instrument, and a robot.

11. The method of claim 1, wherein the step of cutting around the periphery of the targeted tissue is accomplished using an endoscopic cutting instrument,

12. The method of claim 11, wherein the step of cutting around the periphery of the targeted tissue uses a cutting instrument with a hollow end or ceramic tip in order to assist in cutting only to the predetermined depth.

13. The method of claim 1, wherein the step of removing the targeted tissue includes using a retrieval device to engage the targeted tissue.

14. The method of claim 13, wherein the retrieval device is one selected from the group of a snare and forceps.

15. The method of claim 1, further comprising the step of removing the endoscope after deployment of the T-anchor and loop anchor followed by reintroducing the endoscope proximate to the targeted tissue.

16. A medical device to lift targeted tissue, the device comprising:
a suture strand having a distal and proximal end;
a first anchor member having a bar fixed to the distal end of the suture strand;
a second anchor member having a bar and a flexible loop projecting away from the bar;
a flexible delivery needle defining at least one passageway sized to slidably receive the suture strand, the first anchor member, and the second anchor member; the first anchor member positioned distal to the second anchor member within the needle, the suture strand passing through the loop of the second anchor member within the needle; and
a stylet sized to be slidably received by the delivery needle; the stylet being in communication with the second anchor member such that the stylet can cause the first anchor member and then the second anchor member to exit the needle in order for the physician to couple the first anchor member to the targeted tissue;
wherein when tension is applied to the suture strand, the suture strand slides through the loop of the second anchor member to move the first anchor member.

17. The medical device of claim 16, wherein the device is further characterized by a housing that can be manipulated by the physician and has at least one passageway sized to slidably receive the delivery needle.

18. The device of claim 16, wherein the device is further characterized by a cutting instrument sized to be slidably received in one passageway of either the needle or the housing;
wherein the cutting instrument is capable of being manipulated by the physician to separate the targeted tissue from other tissue.

19. The device of claim 16, wherein the loop anchor comprises a crossbar and a strand, the crossbar having first and second opposing ends and defining a longitudinal axis, the crossbar being defined by a tubular wall having an aperture between the first and second ends, the strand having first and second opposing ends connected to the first and second opposing ends of the crossbar, respectively, the strand making a revolution to define a loop, the strand and its loop projecting through the aperture and away from the longitudinal axis, the loop sized to slidably receive the suture therein and protect the suture from abrasion against the crossbar;
wherein the strand has a sufficient length to size the loop to project through the tissue in which the loop anchor is embedded.

20. The medical device of claim 16, wherein the device further comprises:
a retrieval device sized to be slidably received by a passageway of the housing; the retrieval device capable of engaging the targeted tissue to further separate the targeted tissue from other tissue.
